(19) United States
(12) Patent Application Publication
(10) Pub. No.: US 2009/0216334 A1
(43) Pub. Date: Aug. 27, 2009

(54) Bone Implants

(75) Inventor: David A. Leibel, Princeton, MN (US)

Correspondence Address: MORGAN, LEWIS & BOCKIUS LLP, 1701 MARKET STREET, PHILADELPHIA, PA, 19103-2921, US

(73) Assignee: SMALL BONE INNOVATIONS, INC., New York, NY (US)

(21) Appl. No.: 11/884,963

(22) PCT Filed: Feb. 23, 2006
(86) PCT No.: PCT/US06/06587

§ 371 Date: Nov. 3, 2008

Related U.S. Application Data
(60) Provisional application No. 60/655,316, filed on Feb. 23, 2005.

(51) Int. Cl. A61F 2/42

(52) U.S. Cl. 623/21.18

(57) ABSTRACT

The present invention includes implants that include a nickel-titanium alloy. The present invention includes a fusion block implant that includes a nickel-titanium alloy. The present invention includes a wedge, osteotomy implant that includes a nickel-titanium alloy. The present invention includes a subtalar implant that includes a nickel-titanium alloy. The present invention includes methods of bone fusion, and methods of correcting directionality of a bone.

### BACKGROUND OF THE INVENTION

Fusion Implant

There are many examples of fusion implants, in particular wrist fusion implants, currently available on the market today include plates, staples, screws, and wires. These devices are generally mounted within the body to bridge two bones to be fused together. For example, in a radio-carpal fusion, a plate may be mounted on the dorsal (top) side of the wrist, bridging the radius and the carpals of the hand.

Osteotomy Wedge Implant

An osteotomy requires the cutting of bone, usually to correct a defect in the directionality of the bone. Corrective osteotomies can be performed on any long bone in the body. Some examples of corrective osteotomies include tibial osteotomies, femoral osteotomies, ulnar shortening, radial osteotomies, and bunionectomies. For example, in a traditional bunionectomy, a wedge of bone is cut out from the side opposite the bunion, the bone is re-aligned, packed with bone graft, and then left to fuse back together. Sometimes, if adequate fixation of the bone graft material is not achieved during the osteotomy, or if fixation becomes compromised during the healing process, bone resorption can occur and will result in a non-union, causing the patient pain and requiring additional surgery.

Subtalar Implant

Currently, there are a variety of subtalar devices available, including the MBA Screw from KMI Incorporated, the Conical Subtalar Implant from Futura Biomedical, and the Sta-Peg from Wright Medical, Inc. Subtalar implants are used for various indications, including calcaneal valgus deformity, plantar-flexed talus, severe pronation, flatfoot deformity, post tarsal coalition repair, subtalar instability, and supple deformity in posterior tibial tendon dysfunction.

Materials Useful for Making Implants

Nickel-titanium alloys are known shape memory alloys which have been proposed for use in various environments including robotics and in memory devices of medical implants.

U.S. Pat. Nos. 4,206,516; 4,101,984; 4,017,911 and 3,855,638 all describe composite implants having a solid substrate with a thin porous surface coating. U.S. Pat. No. 3,852,045 describes a bone implant element of porous structure in which the pores are developed by means of solid expendable void former elements which are arranged in a selected spatial pattern in a form cavity; metallic particles are packed about the void former elements, the mix is densified, the void former elements are removed, such as by vaporization and the metallic particles are sintered.

### SUMMARY OF THE INVENTION

In an embodiment of the present invention, a fusion block implant is described. In one embodiment, the implant includes a plurality of voids for engaging a fixation device, and the fusion block implant is configured to fit at an interface of a first bone and a second bone. In one embodiment, the fusion block implant includes nitinol. In one embodiment, the nitinol is porous. In another embodiment, the fusion block implant is coated with porous nitinol. In another embodiment, the porosity of the nitinol is from about 20% to about 80%, and in another embodiment, about 65%.

In an embodiment of the present invention, an osteotomy implant is described. In an embodiment of the invention, the osteotomy implant is shaped as a wedge, and in an embodiment, the osteotomy implant is configured to fit between a first surface and a second surface of a bone in need of directionality correction. In another embodiment, the osteotomy implant includes nitinol. In one embodiment, the nitinol is porous. In another embodiment, the osteotomy implant is coated with porous nitinol. In another embodiment, the porosity of the nitinol is from about 20% to about 80%, and in another embodiment, about 65%.

In another embodiment of the invention, a subtalar or small foot or ankle bone implant is described. In an embodiment, the subtalar or small foot or ankle bone implant is non-threaded. In another embodiment, the subtalar or small foot or ankle bone implant is cylindrical, as shown in FIG. 6. In another embodiment, the subtalar or small foot or ankle bone implant includes nitinol. In one embodiment, the nitinol is porous. In another embodiment, the subtalar or small foot or ankle bone implant is coated with porous nitinol. In another embodiment, the porosity of the nitinol is from about 20% to about 80%, and in another embodiment, about 65%.

In another embodiment of the invention, a method for fusing one or more bones is described. In an embodiment of the invention, the method includes separating one or more bones in need of fusing, for example, by cutting. In an embodiment of the invention, the method includes placing a bone fusion implant between a first surface and a second surface of one or more bones. In another embodiment, the method includes securing the implant to one or more bones. In another embodiment, the method includes securing the implant to one or more bones using bone screws. In another embodiment, the method includes allowing bone to grow on and into the implant. In an embodiment of the invention, the implant includes nitinol. In one embodiment, the nitinol is porous. In another embodiment, the implant is coated with porous nitinol. In another embodiment, the porosity of the nitinol is from about 20% to about 80%, and in another embodiment, about 65%. In an embodiment of the invention, the implant is a fusion block implant. In another embodiment, the implant is an osteotomy implant.

In another embodiment, a method for correcting the directionality of a bone is described. The method includes separating a bone, for example, by cutting. In another embodiment, the method includes placing an implant comprised of porous nitinol between a first surface and a second surface of the bone. In another embodiment, the implant is wedge-shaped. In another embodiment, the method includes allowing bone to grow over and into the implant.

### BRIEF DESCRIPTION OF DRAWINGS

FIG. 1, comprising FIGS. 1A-1G, illustrates a radio-carpal fusion block according to the present invention. FIG. 1A is a front view. FIG. 1B is a side view. FIG. 1C is a left isometric view. FIG. 1C is a right isometric view. FIG. 1E is a cross-sectional view of one of the voids. FIG. 1F is a right isometric view depicting the texture of nickel-titanium alloy. FIG. 1G illustrates one embodiment of the present invention, where the fusion implant is configured to fit at the interface between the radial and carpus bones.

FIG. 2, comprising FIGS. 2A-2G, illustrates an osteotomy wedge according to the present invention. FIG. 2A is a top view. FIG. 2B is a side view. FIG. 2C is a left isometric view. FIG. 2D is a right isometric view. FIG. 2E is a right isometric view depicting the texture of nickel-titanium alloy. FIG. 2F illustrates one embodiment of the present invention, where the wedge implant is used to reposition the metatarsal in a bunionectomy. FIG. 2G illustrates another embodiment of the present invention, where the wedge implant is used to reposition both the metatarsal and the phalangeal bones in the bunionectomy.

FIG. 3, comprising FIGS. 3A-3F, illustrates a subtalar or small foot or ankle bone implant according to the present invention. FIG. 3A is a top view. FIG. 3B is a side view. FIG. 3C is a left isometric view. FIG. 3D is a right isometric view. FIG. 3E is a cross-sectional view. FIG. 3F is a right isometric view depicting the texture of nickel-titanium alloy.

FIG. 4 is an SEM of a rough surface of a porous nitinol material useful in the present invention.

FIG. 5 is an SEM of a smooth surface of a porous nitinol material useful in the present invention.

FIG. 6 illustrates a cylindrical implant according to the present invention.

### DETAILED DESCRIPTION

The present invention is directed to new medical implants, particularly bone implants. In one embodiment, the present implants may be configured to fit between two portions of a bone of a person of any size, stature, and bone structure. In an embodiment of the invention, the implants are custom-made to fit each individual. In another embodiment of the invention the implants are made in a range of standard sizes to fit normal anthropomorphic features.

In an embodiment of the invention, the implants are bone fusion implants. In an embodiment of the invention, the implants include nickel-titanium alloy (NiTi; nitinol). In another embodiment, the nitinol alloy is porous. In another embodiment, the NiTi is porous. In another embodiment, the NiTi porosity is between 0% and 100%. In another embodiment the porosity of the NiTi is between about 20% to about 80%. In another embodiment, the porosity is between about 40% and 70%. In another embodiment, the porosity is about 65%. In another embodiment, the implant is coated with NiTi.

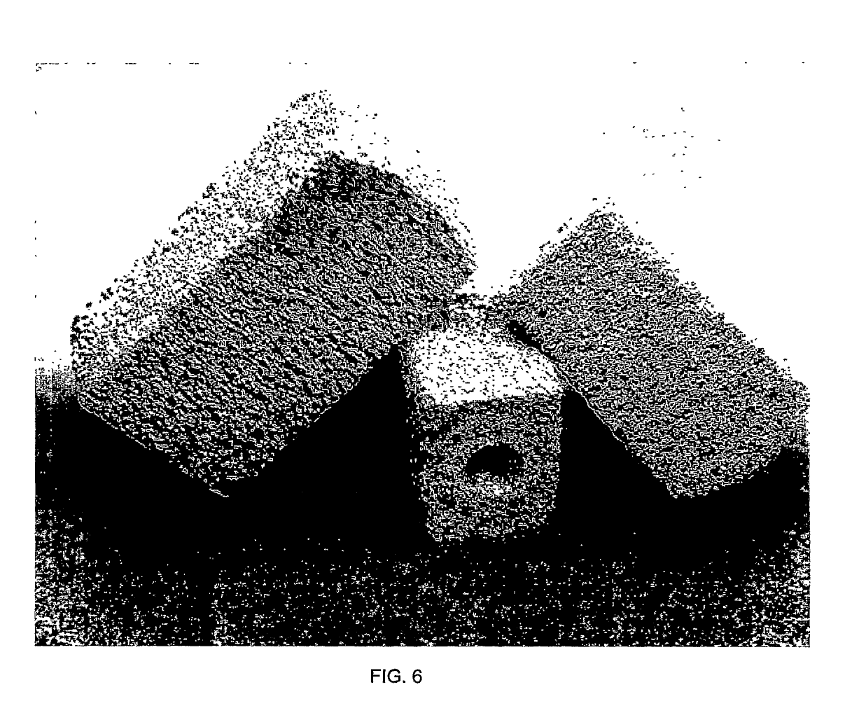

In an embodiment of the present invention, the implants described herein are of any shape necessary to conform to a specific bone to bone interface. In an embodiment of the invention, the implant is round. In another embodiment, the implant is square. In another embodiment, the implant is cylindrical, for example as shown in FIG. 6. In another embodiment, the implant is rectangular. In an embodiment of the invention, the implant is circular or disc-shaped. In another embodiment, the implant is of a regular or irregular shape. In another embodiment, the implant is wedge-shaped. In another embodiment, the implant includes voids. In another embodiment, the implant is threaded. In another embodiment, the implant is non-threaded.

Fusion Block Implant

In one embodiment, the present invention is directed to a fusion implant, whereby bone grows in and on the implant such that the bones between which the implant is placed are fused to one another via the fusion implant. This implant may be configured to be accommodated between two or more bone portions within a person of any size, stature, and bone structure. This implant may be used at any point in the body where two bones require fusion. For example, in an embodiment of the invention, the fusion implant is configured as a wrist or radio-carpal fusion implant, which is placed between the radius and carpal bones, thereby resulting in the fusion of the scaphoid and lunate bones of the hands to the scaphoid and lunate fossae of the distal radius.

In an embodiment of the invention, placing the implant of the present invention between, for example, the radius and carpal bones for wrist fusions, enhances the aesthetic appearance of having an implant because the implant does not protrude under the skin. In addition, mounting the implant at the interface of the two bones minimizes the risk of irritation or rupture of the extensor tendon in the hand. Another advantage of the wrist fusion implant of the present invention is a decrease in the likelihood of a failed fusion (or “non-union”), and a decrease in the likelihood of loss of fixation of the device during the healing process due to the rotational torque of the wrist.

In an embodiment of the invention, the fusion implant has at least one void and preferably a plurality of voids that cut through at least two faces of the fusion implant. The voids may be of varying size to accommodate a fixation device, for example, a screw, to fix the fusion implant to the bones requiring fusion, or to those bones surrounding the bones requiring fusion. Other fixation devices include a wire, a post, a staple, a plate, and a peg, such fixation devices engaging the implant and a bone to hold the implant in place. In an embodiment of the invention, the voids are cut straight through the fusion implant, perpendicular to the faces of the implant being cut. In another embodiment, the voids are angled upward or downward. In another embodiment, the voids are angled outward toward the edge of the implant or inward toward the middle of the implant. The angle of the voids is changed as necessary to accommodate any anatomical region of the interface between the bones requiring fusion. The angle can be between 0 and 90 degrees relative to a face of the fusion implant.

In an embodiment of the invention, the fusion implant has from about 2 voids to about 8 voids. In an embodiment of the invention, the voids are spaced uniformly between each other. In another embodiment of the invention, the voids are spaced non-uniformly as required for placement between two or more bone portions. In another embodiment, the voids are spaced such that the implant will be in contact with a bone upon engagement of a fixation device, for example, a screw. The diameter of the voids may be the same or different, depending on the size of the required fixation device to be inserted through the fusion implant. The choice of fixation device will depend on the size, density, and location of the bones requiring fusion, and will also depend on the type of fixation required.

In an embodiment of the present invention, the fusion implant is any shape or size depending on the shape and size of the two or more bone portions to be fused together. In one embodiment, the fusion implant is rectangular in shape. In another embodiment, the fusion implant is oval in shape. In another embodiment, the fusion implant is square. In another embodiment, the fusion implant is spherical. In another embodiment, as shown in FIG. 6, the fusion implant is cylindrical. In another embodiment, the fusion implant is conical. The length, width, and height of the fusion implant will vary depending on the size of the implant necessary to maximize bone fusion or depending on the anatomical location of the bones to be fused.

In an embodiment of the invention, the implant includes nickel-titanium (NiTi) alloy. In another embodiment, the NiTi alloy is porous. In another embodiment, the NiTi porosity is between 0% and 100%. In another embodiment the porosity of the NiTi is between about 20% to about 80%. In another embodiment, the porosity is between about 40% and 70%. In another embodiment, the porosity is about 65%. In another embodiment, the implant is coated with NiTi alloy.

Example of Specific Embodiment of Wrist Fusion Implant

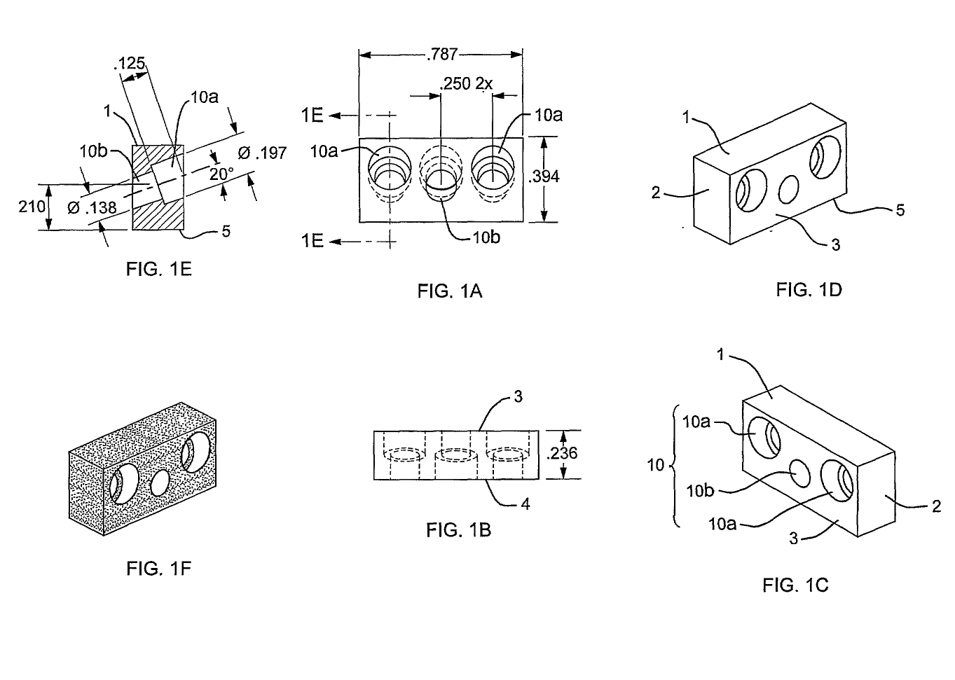

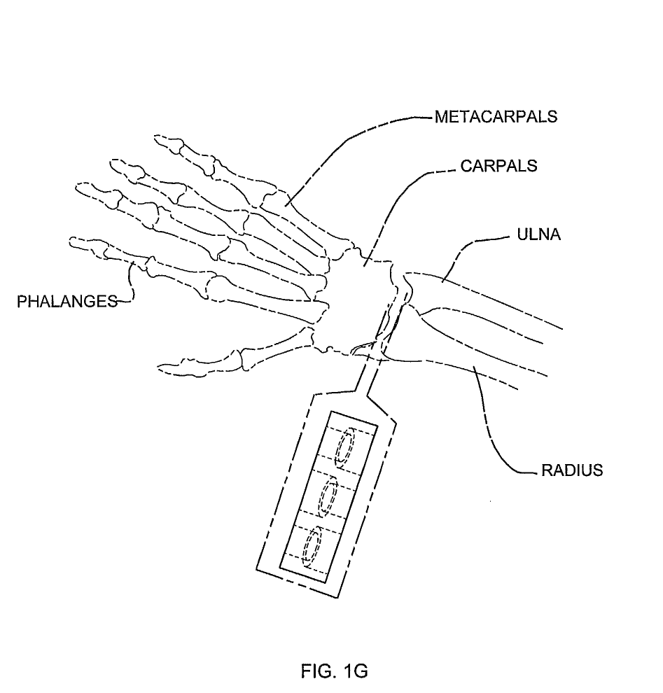

In an embodiment of the present invention, the fusion implant is a rectangular radio-carpal fusion implant as described in FIGS. 1A-1G. In an embodiment of the invention, voids 10 are configured in accordance with FIG. 1A, where voids 10a are of the same size and are at each end of the implant, with a smaller void 10b in the middle and slightly lower than the other two voids 10a. In an embodiment of the invention, as shown in FIGS. 1A and 1E, voids 10a cut through the implant front face 3 at a downward angle of about 20 degrees and void 1Ob cuts through implant front face 3 at an upward angle of about 20 degrees. In one embodiment, the diameter of voids 10a on implant front face 3 is about 0.197 inches. In one embodiment, the diameter of void 10b on implant front face 3 is about 0.138 inches. In one embodiment, voids 10 have a larger diameter on one face of the implant and a smaller diameter on another face of the implant. For example, in one embodiment, the diameter of voids 10a on the implant back face 4 is about 0.138 inches, and the diameter of void 10b on implant back face 4 is about 0.197 inches. In an embodiment of the invention, voids 10a engage the head of a fixation device, for example, a screw, and void 1Ob engages the bottom of a fixation device, for example, a screw.

In another embodiment of the invention, voids 10 cut through implant front face 1 and one of the implant side faces 2. In another embodiment, voids 10 cut through implant top face 1 and one of implant side faces 2. In another embodiment, voids 10 cut through implant top face 1 and the implant bottom face 5. In yet another embodiment, voids 10 cut through at least two of implant top face 1, implant bottom face 5, implant front face 3, implant back face 4, and implant side faces 2.

In an embodiment of the invention as shown in FIGS. 1A and 1B, the width of the implant is about 0.236 inches, the height of the implant is about 0.394 inches, the length of the implant is about 0.787 inches, and the space between the midpoints of voids 10a and 10b is about 0.250 inches. In an embodiment of the invention, a 3 mm flat-bottom cancellous screw is useful as a fixation device.

In another embodiment of the invention, as shown in FIG. 1G, a rectangular section is cut out of the radio-carpal interface, and the implant is placed in the joint space between the proximal carpal row and the distal radius.

In another embodiment of the invention, as shown in FIG. 1F, the fusion implant includes nickel-titanium alloy. In one embodiment, the alloy is coated on the entire fusion implant. In another embodiment, the fusion implant is made from the alloy. In another embodiment, one or more portions of the fusion implant includes the alloy.

Osteotomy Wedge Implant

In an embodiment, the present invention also includes a wedge-shaped implant useful for corrective osteotomies.

In an embodiment of the present invention, the wedge implant is used in a bunionectomy. When performing a bunionectomy using the implant of the present invention, a transverse cut is made through the bone on the same side as the bunion, the bone may or may not be repositioned, and the wedge-shaped implant of the present invention is pressed between the cut portions of the bone, thereby straightening the bone. Thus, one embodiment of the present invention reduces the risk of non-union, thereby reducing the risk of bone resorption, pain, and additional surgery.

In another embodiment of the invention, the wedge is used for a radial osteotomy at the distal radius of the hand.

In an embodiment of the invention, the wedge is used in conjunction with plates, screws, wires, or other fixation devices to provide temporary fixation of the wedge during rehabilitation.

The size and shape of the wedge will depend on the size of the bone on which the wedge is to be used and the severity of the required repositioning of the bone. For example, a wedge useful for a radial osteotomy will be larger in size than a wedge useful for a bunionectomy. Likewise, a wedge useful for a mild repositioning of the bone will be of a different size and shape than a wedge useful for a severe repositioning of the bone. In another embodiment, one or more wedges can be used to reposition a bone.

In an embodiment of the present invention, a set of standard wedges is created for each bone on which the wedge will be used to accommodate normal anthropomorphic features. In another embodiment, the wedge is custom-made to accommodate those falling outside the normal anthropomorphic features.

In an embodiment of the present invention, the faces of the wedge implant are any shape or size depending on the shape and size of the bone on which it is being used. In one embodiment, at least two faces of the wedge implant are rectangular in shape. In another embodiment, at least two faces of the wedge implant are oval in shape. In another embodiment, at least two faces of the wedge implant are square. In another embodiment, at least two faces of the wedge implant are circular. In another embodiment, at least two faces of the wedge implant are triangular. The length, width, and height of the wedge implant will vary depending on the size of the implant necessary to maximize successful repositioning of the bone.

The degree of angulation of the wedge will depend on the severity of the required positional correction. In one embodiment, the wedge is angled at an angle of from about 0 degrees to about 60 degrees. In another embodiment, the wedge is angled at an angle of from about 10 to about 40 degrees. In another embodiment, the wedge is angled at an angle of about 20 degrees.

In an embodiment of the invention, the implant includes NiTi alloy. In another embodiment, the NiTi alloy is porous. In another embodiment, the NiTi porosity is between 0% and 100%. In another embodiment the porosity of the NiTi is between about 20% to about 80%. In another embodiment, the porosity is between about 40% and 70%. In another embodiment, the porosity is about 65%. In another embodiment, the implant is coated with NiTi alloy.

Specific Embodiment of Osteotomy Wedge Implant

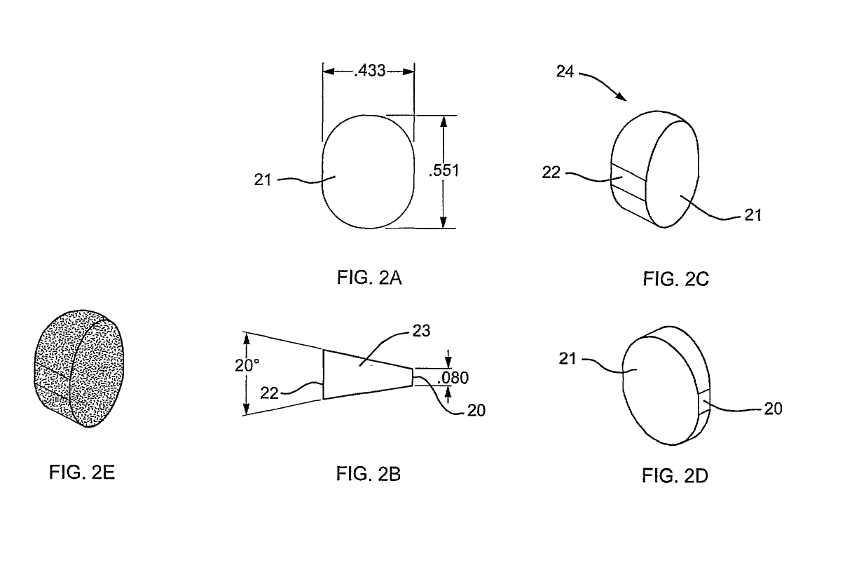

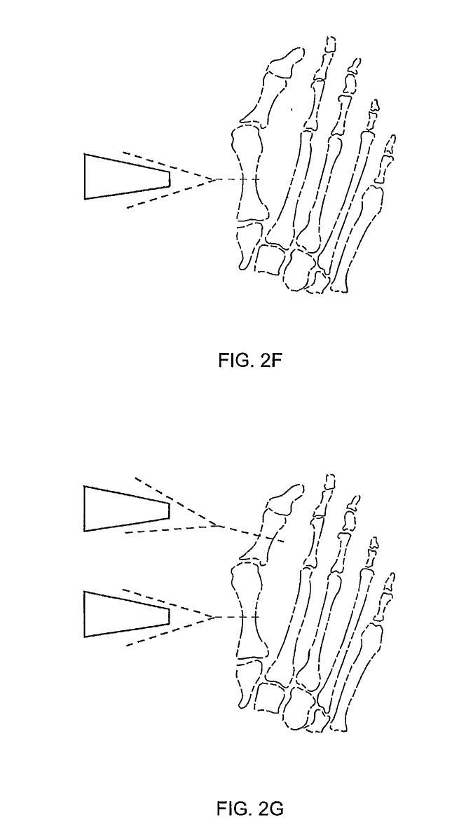

In one embodiment of the present invention, as shown in FIGS. 2F and 2G, the wedge is used in a bunionectomy procedure to reposition the metatarsal bone. In one embodiment, a transverse cut is made through the metatarsal bone, the bone is repositioned, and the wedge is inserted as indicated in FIG. 2F. As shown in FIGS. 2A-2D, the wedge implant is about 0.551 inches in length, 0.433 inches wide, and wedged at a 20 degree angle. The base of the wedge is about 0.080 inches. In an embodiment of the invention as depicted in FIGS. 2A-2D, the top face 21 and the bottom face 24 are oval, the front face 20 and the back face 22 are rectangular, and the side faces 23 are trapezoidal in shape. In another embodiment of the invention, top face 21 and bottom face 24 are round. In another embodiment, top face 21 and bottom face 24 are square. In another embodiment, top face 21 and bottom face 24 are rectangular. In another embodiment, top face 21 and bottom face 24 are trapezoidal. In another embodiment, top face 21 and bottom face 24 are triangular.

In another embodiment of the invention, as shown in FIG. 2E, the wedge implant includes nickel-titanium alloy. In one embodiment, the alloy is coated on the entire wedge implant. In another embodiment, the wedge implant is made from the alloy. In another embodiment, one or more portions of the wedge implant include the alloy.

In another embodiment of the invention, as illustrated in FIGS. 2F and 2G, the wedge is shown being used in a bunionectomy. In another embodiment, the wedge osteotomy implant is used to correct the directionality of a bone. In an embodiment of the invention, the wedge osteotomy implant is configured to fit at an interface between two bone portions, such that the implant corrects the directionality of a bone.

Subtalar or Small Foot or Ankle Bone Implant

The present invention is also directed to a subtalar implant, and small foot and ankle bone fusion implants. The subtalar and small foot or ankle bone fusion implants of the present invention enhances the ability of the implant to remain fixed, and decreases the likelihood that the implant will loosen or “back out” over time.

In an embodiment of the present invention, the size of the subtalar and small foot or ankle bone fusion implants will depend on the size of the bone on which the implant is to be used. In an embodiment of the present invention, standard sizes of implants are created that will accommodate normal anthropomorphic features. In another embodiment, the implant is custom-made to accommodate those falling outside the normal anthropomorphic features.

In an embodiment of the invention, the subtalar and small foot or ankle bone fusion implants are non-threaded. In another embodiment, the subtalar or small foot or ankle bone fusion implants are cylindrical (as shown in FIG. 6) or conical in shape.

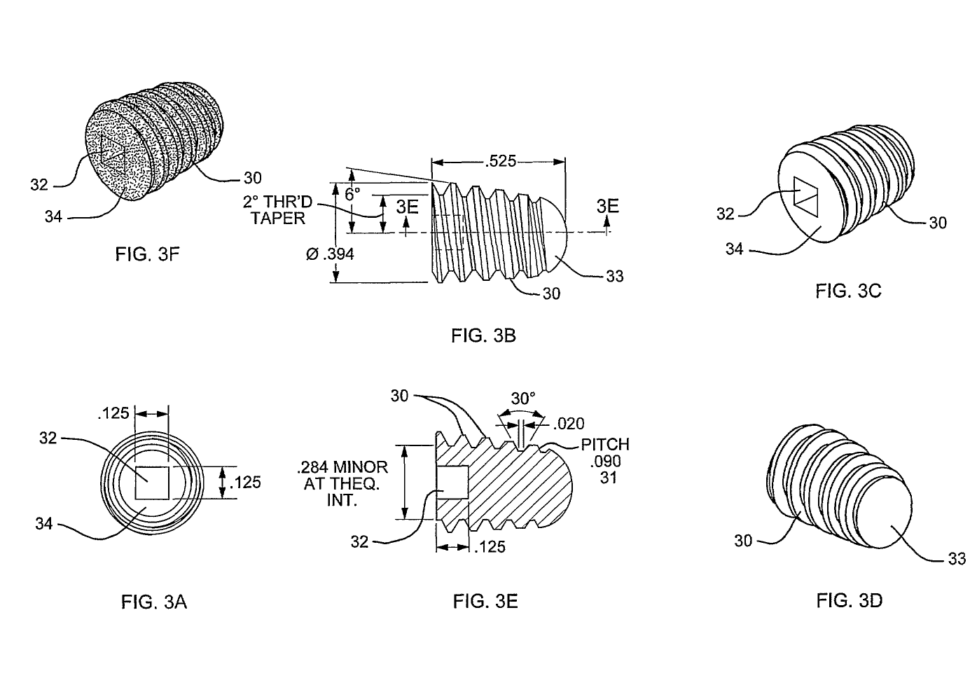

In an embodiment of the invention as set forth in FIGS. 3A-3F, the subtalar or small foot or ankle bone fusion implant are threaded. In an embodiment of the invention, the threads 30 have a pitch 31 of between about 0 and about 0.3 inches. In another embodiment, pitch 31 is from about 0 to about 0.1 inches.

In an embodiment of the invention, the distance between threads 30 is between about 0 and about 0.1 inches. In an embodiment of the invention shown in FIG. 3, the space between threads 30 is flat. In another embodiment, the space between threads 30 is rounded or curved.

As shown in FIGS. 3B and 3C, in one embodiment, a subtalar implant according to the present invention is about 0.525 inches in length, 0.394 inches in diameter, and has a thread pitch 31 of about 0.090 inches. Each thread 30 is spaced about 0.020 inches apart, and the angle between each of threads 30 is about 60 degrees. In an embodiment of the invention, the size of an implant according to the present invention will depend, in part, on the size, stature, and bone structure of the individual using the implant. Also shown in FIGS. 3B and 3C, in one embodiment, the interior width of a subtalar implant according to the present invention is about 0.284 inches, while the exterior width is about 0.394 inches. In an embodiment of the invention, the interior and exterior width measurements will change based on pitch 31 of threads 30.

In another embodiment, the angle between each of threads 30 of the implant is from about 0 to about 90 degrees. In another embodiment, the angle is about 60 degrees.

With reference to FIGS. 3A-3F, in an embodiment of the present invention, the implant includes a drive mechanism 32 for engagement by a screwdriver or other tool to drive the implant into the bone. In an embodiment of the invention, drive mechanism 32 is configured to engage a tool for driving the implant into the bone. In an embodiment of the invention, the base 34 of the implant is configured to include drive mechanism 32.

In an embodiment of the present invention, the tip 33 of the implant is rounded or blunt. In another embodiment, tip 33 is pointed or sharp.

In an embodiment of the invention, as shown in FIG. 3F, the implant includes NiTi. In one embodiment, the NiTi is coated on the entire implant. In another embodiment, the implant is made from NiTi. In another embodiment, one or more portions of the subtalar or small foot or ankle bone implants include the NiTi.

In another embodiment, the NiTi alloy is porous. In another embodiment, the NiTi porosity is between 0% and 100%. In another embodiment the porosity of the NiTi is between about 20% to about 80%. In another embodiment, the porosity is between about 40% and 70%. In another embodiment, the porosity is about 65%.

In an embodiment of the invention, the implant is threaded between the talus and the calcaneous bones of the ankle.

Materials Useful for Making the Implants Described Above

In an embodiment of the present invention, the implants discussed above are prepared from stainless steel. In another embodiment of the present invention, the implants discussed above are prepared from titanium. In another embodiment of the present invention, the implants discussed above are prepared from cobalt chrome. Other materials, such as plastics, polymers, and other metals are also useful in the present invention. Combinations of materials are also useful in the present invention, for example, a combination of plastic and nickel-titanium alloy. Any material used in the orthopedic industry to make implants is useful in the present invention.

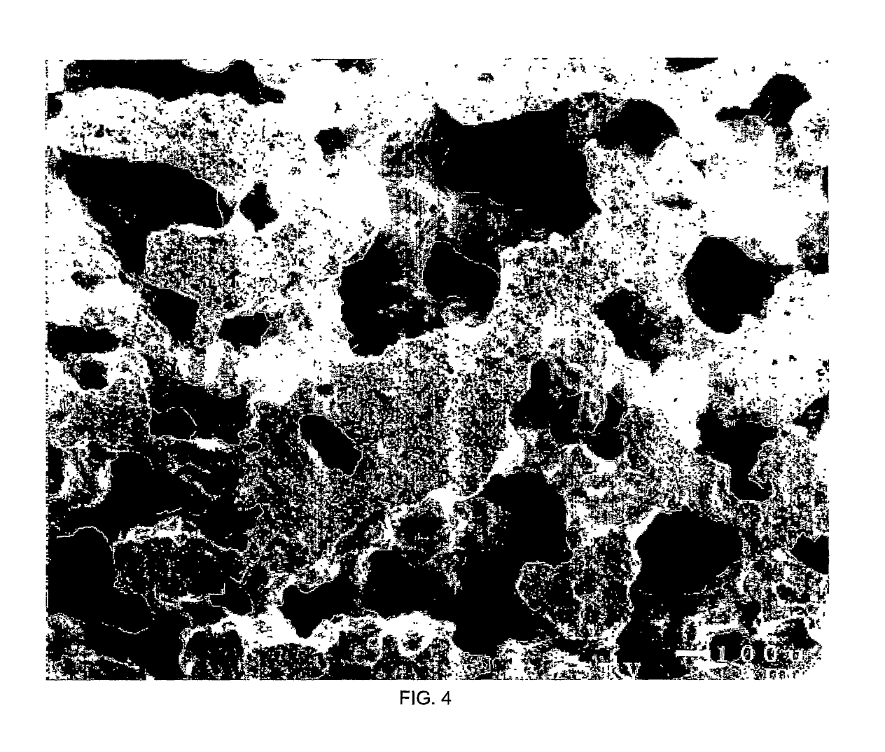

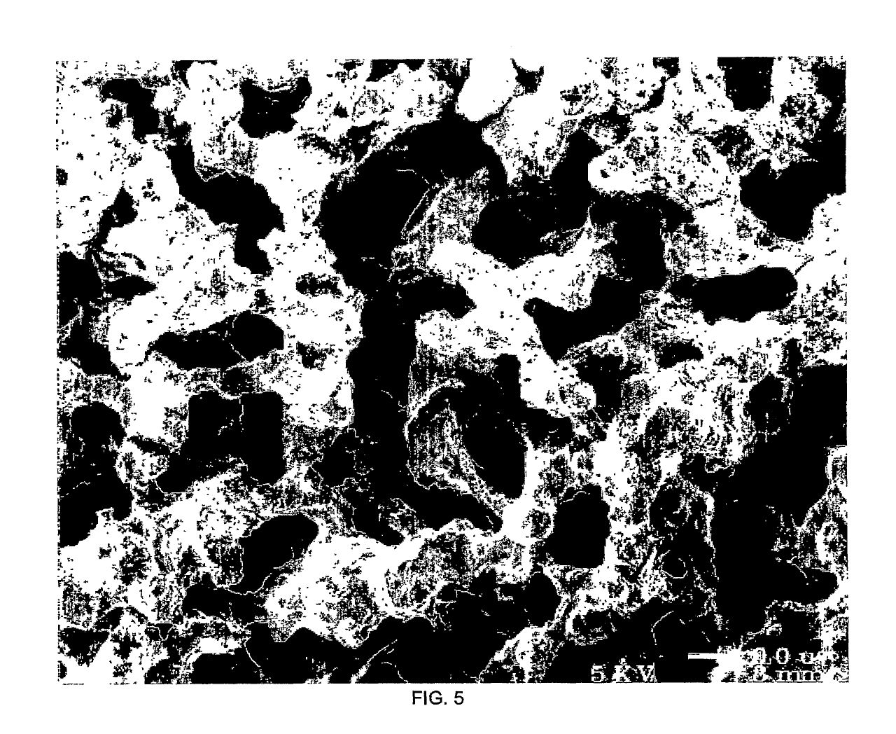

In an embodiment of the present invention, the implants described above are prepared from a porous nickel-titanium alloy (nitinol or NiTi) as described in U.S. Pat. No. 5,986,169, herein incorporated by reference in its entirety. By way of example, FIGS. 4 and 5 illustrate porous nitinol compositions. The nickel-titanium alloy described therein has a porosity of 8 to 90% and the porosity is defined by a network of interconnected passageways, the network exhibiting an isotropic permeability for fluid material effective to permit complete migration of the fluid material throughout said network.

Nickel-titanium alloy has certain advantages, as compared with other materials, in biomedical applications. In particular, it displays a high level of inertness or biocompatibility, and it has high mechanical durability, thus providing longevity when employed in the fabrication of implants. This is advantageous because live tissue has an elasticity which renders it resilient to permanent deformity when subjected to stress and vibrations. Therefore, if the material used to produce an implant that contacts live tissue has different characteristics from the tissue, it will not meet the requirement for biocompatibility in an implant and longevity will be short. Also, the osseo-integrative properties of this alloy may promote superior fusion as compared to other grafting substitutes and/or other implant materials, such as stainless steel or titanium, when used for bone implants. Nickel-titanium alloy displays mechanical behavior very similar to that of live tissue, thus showing high biocompatibility.

Nickel-titanium alloys have a high level of biocompatibility with human tissue and the capillarity of this material facilitates penetration of the material by human biological fluids under the force of capillary action. Thus, biological fluid from the bone is drawn into the network of passageways of the contacting tissue, and the fluid migrates, under capillary action, throughout the network. Live tissue in the fluid grows within the pores of the network and adheres to the pore surfaces providing a chemical bonding or unification with the nitinol. As growth of tissue, for example, bone, is completed there is provided both a chemical bonding between the newly grown bone and the nitinol material.

In an embodiment of the present invention, the nitinol material is fabricated as an implant or endoprosthesis for local or total replacement of a body part, for example, to correct birth defects or defects resulting from injury or disease.

In another embodiment, nitinol is fabricated as a spacer to replace a portion of shattered human bone and to provide a bridge for connection of bone parts separated as a result of the shattering of the original bone.

In another embodiment, nitinol is fabricated as a fusion implant to provide structure, support and an environment for fusion of adjacent bone surfaces.

In an embodiment of the invention, the porous nickel-titanium alloy comprises 2 to 98% by weight titanium and 98 to 2% by weight nickel, to a total of 100%. In another embodiment, the porous nickel-titanium alloy comprises 40 to 60% by weight titanium and 60 to 40% by weight nickel, to a total of 100%.

In one embodiment of the invention, the implant includes a nickel-titanium alloy having a porosity of at least 40% and not more than 80%. In another embodiment of the invention, the implant includes a nickel-titanium alloy having a permeability derived from capillarity in the network of passageways which define the porosity. Capillarity may be produced in the implant by inclusion of a large number of pores of fine size which interconnect to produce capillary passages.

In an embodiment of the present invention, capillarity is advantageous because it promotes migration of a desired fluid material into the network of passageways, and retention of the fluid material in the network, without the need to apply external hydraulic forces. In an embodiment of the invention, the network of passageways has a coefficient of permeability of 2×10⁻¹³ to 2×10^{-5,} and the permeability is isotropic. In an embodiment of the invention, the capillarity and the isotropic character are, in particular, achieved when the network defining the porosity includes pores of different sizes. In an embodiment of the present invention, the pore size distribution is as follows:

| Pore Size in Microns | Quantity |
|---|---|
| 10⁻²-10 | 1-15% |
| 10⁻¹-10 | 5-10% |
| 10-100 | 10-20% |
| 100-400 | 20-50% |
| 400-1000 | 10-50% |
| above 1000 | remainder to 100% |

In an embodiment of the present invention, the porosity of the nickel-titanium alloy affects its physio-mechanical qualities, for example, mechanical durability, corrosion resistance, super-elasticity and deformational cyclo-resistivity.

In another embodiment of the invention, the size of the pores, the directional penetrability and the coefficient of wettability for biological fluids, as well as factors such as differential hydraulic pressure in the saturated and unsaturated medical implant that includes the nickel-titanium alloy, determine the speed and adequacy of penetration of a biological fluid into the medical implant that includes the alloy.

In another embodiment of the invention, pore size is also an important factor in tissue or biological aggregate growth. At least some of the pores need to be of a size that permits development or growth of biological aggregates synthesized from the components of the fluid, for example, osteons, in the case of bone tissue.

In another embodiment of the invention, optimal pore size will provide permeability to the biological fluid and effective contact for bonding of components in the fluid with the interior pore surfaces of the medical implant. The area of these surfaces depends on the pore sizes and the pore size distribution.

In an embodiment of the present invention, if the pore size of the nickel-titanium alloy is decreased, the permeability changes unpredictably, since the hydraulic resistance increases while the capillary effect appears at a certain low pore size, which capillary effect increases the permeability.

In another embodiment, if pore size is increased, the capillary effect decreases and the durability of the porous article also decreases. For each kind of live tissue there are optimum parameters of permeability, porosity and pore size distribution for efficient operation of the medical implant. The nickel-titanium alloy functions well with a wide variety of live tissue, including bone, and thus permits wide scope of use.

In an embodiment of the present invention, the nickel-titanium alloy permits a wide field of application, including medical implants, without modifying the biomechanical and biochemical compatibility.

In an embodiment of the invention, the nitinol material is Actipore™ from Biorthex, Inc. (Quebec, Canada). Actipore™ is a porous nitinol (TiNi), which is an intermetallic TiNi molecule with excellent biological and biomechanical compatibility.

Actipore™ is a porous, biologically and biomechanically compatible nitinol material, having a porous structure made of interconnected passageways which permit bone cell penetration, long term bone cell survival and integration throughout the devices.

In an embodiment of the invention, the Actipore™ material, as a consequence of the isotropic interconnected porous structure and the capillary wicking forces, actively draw essential fluids and nutrients into the implant allowing for strong, rapid growth of newly forming bone cells throughout its ultra porous scaffold. Thus, in an embodiment of the invention, as a result of these forces, no additional bone graft material is required, thus eliminating the risk of associated graft site morbidity.

In an embodiment of the invention, the Actipore™ material has a low modulus of elasticity, closely resembling that of cancellous bone. In another embodiment, the Actipore™ material is compatible with MRI and CT scans.

In an embodiment of the invention, the Actipore™ material has an approximate porosity of about 65% and an average pore size of about 215 microns, resulting in immediate perfusion and strong rapid growth of newly forming bone throughout its ultra porous scaffold. Although porous, the Actipore· material has increased compressive strength in comparison to bone, while sharing a similar modulus of elasticity, therefore minimizing the risk of stress shielding and the risk of compromising performance of a device made from this material.

Process for Making Nickel-Titanium Alloy

The porous article is produced with a controlled pore size distribution, as indicated above. In particular the porous article may be produced in accordance with the procedures described in the Russian publication “Shape Memory Alloys in Medicine”, 1986, Thompsk University, p-205, Gunther V. et al, the teachings of which are incorporated herein by reference in their entirety. FIGS. 4 and 5 illustrate examples of porous nitinol made by this process. In one embodiment, there is employed the so-called SBS method in which the alloy is produced by means of a layered combustion which exploits exothermic heat emitted during interaction of different elements, for example, metals. In this interaction a thermo-explosive regime takes place. The porosity and porosity distribution are controlled by adjustment of the process parameters.

Nickel-titanium alloys may also be produced in accordance with the disclosure in U.S. Pat. Nos. 4,654,092 and 4,533,411, all of which are hereby incorporated by reference in their entirety.

Methods of Using Bone Implants

Several methods of using the bone implants of the present invention are described in the present application. In an embodiment of the present invention, a method for fusing bone is described. In one embodiment, the method includes separating one or more bones, for example, by cutting, and inserting an implant configured to fit between one or more bone portions of the present invention between the cut surfaces of the bone or bones. In an embodiment of the invention, the implant is secured to the bone or bones, with a fixation device, for example, a bone screw.

In an embodiment of the invention, the implant is secured to the bone or bones. Devices for securing an implant of the present invention to the bone or bones are disclosed elsewhere herein.

In another embodiment, a method for correcting the directionality of a bone is described. The method includes separating a bone, for example, by cutting. In an embodiment of the invention, the method includes implanting an implant of the present invention between a first surface and a second surface of the bone. In an embodiment of the invention, the implant used is irregularly shaped, such that one side of the implant is higher than the other side, for example, a wedge shape.

All patents, applications, and other publications are hereby incorporated by reference in their entirety.

Although only particular embodiments of the invention are specifically described above, it will be appreciated that modifications and variations of the invention are possible without departing from the spirit and intended scope of the invention. Thus, it is intended that the present invention cover the modifications and variations of the present invention provided they come within the scope of the appended claims and their equivalents.

What is claimed is:

1. A fusion block implant, said implant comprising a plurality of voids for engaging a fixation device, wherein said implant is configured to fit at an interface of a first bone and a second bone, and further wherein said implant is comprised of nitinol.

2. The implant of claim 1, wherein the nitinol is porous.

3. The implant of claim 1, wherein the implant is coated with porous nitinol.

4. The implant of claim 2, wherein said nitinol has a porosity of from about 20% to about 80%.

5. The implant of claim 4, wherein the porosity of said nitinol is about 65%.

6. An osteotomy implant, said implant being wedge-shaped, wherein said implant is configured to fit between a first cut surface and a second cut surface of a bone in need of directionality correction and said implant is comprised of nitinol.

7. The implant of claim 6, wherein the nitinol is porous.

8. The implant of claim 6, wherein the implant is coated with porous nitinol.

9. The implant of claim 7, wherein said nitinol has a porosity of from about 20% to about 80%.

10. The implant of claim 9, wherein the porosity of said nitinol is about 65%.

11. A fusion implant, wherein said implant is cylindrical and comprised of porous nitinol.

12. The implant of claim 11, wherein said implant is a subtalar implant.

13. The implant of claim 11, wherein said implant is an implant for an ankle bone.

14. The implant of claim 11, wherein said implant is an implant for a small bone in a foot.

15. The implant of claim 11, wherein the implant is coated with porous nitinol.

16. The implant of claim 11, wherein said nitinol has a porosity of from about 20% to about 80%.

17. The implant of claim 16, wherein the porosity of said nitinol is about 65%.

18. The implant of claim 11, wherein said implant is non-threaded.

19. A method for fusing one or more bones, said method comprising:
(a) separating said one or more bones in need of fusing; and
(b) implanting a bone fusion implant comprising porous nitinol between a first surface and a second surface of said one or more bones,
thereby fusing said one or more bones.

20. The method of claim 19, further comprising securing said implant to said one or more bones.

21. The method of claim 20, wherein said implant is secured to said one or more bones using one or more bone screws.

22. The method of claim 20, further comprising allowing bone tissue to grow on and into said implant.

23. The method of claim 19, wherein said nitinol has a porosity of from about 20% to about 80%.

24. The method of claim 23, wherein said porosity is about 65%.

25. The method of claim 19, wherein said fusion implant is a fusion block implant.

26. The method of claim 19, wherein said fusion implant is an osteotomy implant.

27. The method of claim 19, wherein said fusion implant is cylindrical.

28. The method of claim 19, further comprising growing bone and tissue over and into said implant.

29. A method for correcting the directionality of a bone, said method comprising:
(a) separating said bone; and
(b) placing an implant comprised of porous nitinol between a first surface and a second surface of said bone, said implant being wedge-shaped,
thereby correcting the directionality of said bone.
